(19) United States
(12) Patent Application Publication
(10) Pub. No.: US 2025/0166223 A1
(43) Pub. Date: May 22, 2025

(54) MEDICAL IMAGE PROCESSING APPARATUS AND METHOD

(71) Applicant: CANON MEDICAL SYSTEMS CORPORATION, Otawara-shi (JP)

(72) Inventors: Paul THOMSON, Edinburgh (GB); Ewan HEMINGWAY, Edinburgh (GB); Ivona VICKOVIC, Edinburgh (GB)

(73) Assignee: CANON MEDICAL SYSTEMS CORPORATION, Otawara-shi (JP)

(21) Appl. No.: 18/513,718

(22) Filed: Nov. 20, 2023

(51) Int. Cl. G06T 7/73; G06T 7/11; G06T 7/62; G16H 30/40

(52) U.S. Cl. CPC G06T 7/74; G06T 7/11; G06T 7/62; G16H 30/40; G06T 2207/10081; G06T 2207/20021; G06T 2207/20081; G06T 2207/20084; G06T 2207/30012; G06T 2207/30036

(57) ABSTRACT

A medical image processing apparatus comprises processing circuitry configured to:
obtain a respective position for each of a plurality of anatomical landmarks in a three-dimensional space;
generate a spline based on the positions for the plurality of anatomical landmarks; use the spline to normalize the positions to obtain one-dimensional data or two-dimensional data;
adjust at least one of the anatomical landmarks based on the one dimensional data or two-dimensional data; and
re-map the adjusting of at least one anatomical landmark to the three-dimensional space.

### FIELD

Embodiments described herein relate generally to a method of, and apparatus for, processing medical image data to locate anatomical landmarks, for example to locate vertebrae of the human spine.

### BACKGROUND

It is known to process medical images to identify anatomical landmarks. An anatomical landmark is usually a well-defined point in an anatomy, for example the human anatomy. Anatomical landmarks may be defined in relation to anatomical structure such as bones, vessels or organs.

Automated landmark detection (ALD) may be used to aid bone diagnosis. ALD may be used to detect bony landmarks surrounding the bone metastatis and lesions in the chest. Each of the vertebrae of the spine may be labelled. The use of ALD may aid diagnosis by improving ease of reading and reporting of scans through efficient and accurate identification of location of bone metastasis using the vertebrae labels.

The natural curvature of the spine can make it difficult to detect anatomical landmarks accurately. The vertebra are commonly used as anatomical landmarks when processing images of the human spine. However, there are multiple vertebra in the human body with few differences in appearance. Neighbouring vertebrae have similar appearance which may lead to difficulty in unique and correct detection of each unique vertebrae. The difficulty in assessing the accuracy of the anatomical landmark can make it difficult to automatically order the vertebrae during post processing. For example, neural networks used in image processing can require consistent features to disambiguate landmarks. Detection of repeated structures may be challenging.

If a detection network cannot distinguish neighbouring vertebrae well, it may fail to detect one or more vertebrae along the spine. It may mis-label the vertebra by placing labels for two or more vertebrae in space occupied by a single vertebra. It may mis-label a plurality of vertebrae with labels that are all offset from ground truth by a common offset amount, for example all offset by one. Such offsetting may create a domino effect of errors.

Accurate automatic labelling may, for example, potentially save radiologists minutes per examination, which may add up to more than an hour per day. Accurate automatic labelling may improve a radiologist's workflow. Identifying vertebrae correctly and labelling with an expected anatomical order may increase confidence of a clinician and aid accurate diagnosis

FIG. 1 shows an example of offsetting, in which vertebrae are incorrectly labelled in a medical image 2. Labels shown in FIG. 1 are applied by a detection network. A first vertebra 10 is correctly identified as T6. A second vertebra 12 should be identified as T7, but instead is missed by the detection network and is not labelled by the detection network. A third vertebra 14 should be identified as T8, but is labelled by the detection network as T7. Similarly, vertebrae 16, 18, 20, 22, 24, 26 should be identified as T9, T10, T11, T12, L1, L2 respectively but instead are labelled by the detection network as T8, T9, T10, T11, T12, L1 respectively. Vertebra 28 should be identified as L3, but the detection network labels it with both L2 and L3 labels.

If an identification is incorrect, the labelling may be less useful and clinicians may lose confidence in the labelling.

### BRIEF DESCRIPTION OF THE DRAWINGS

Embodiments are now described, by way of non-limiting example, and are illustrated in the following figures, in which:

FIG. 1 is an example of a medical image showing mis-labelling;

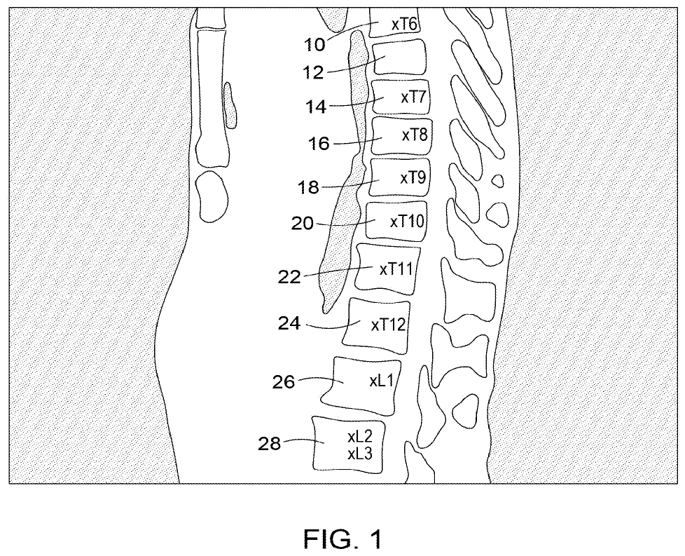

FIG. 2 is a schematic diagram of an apparatus according to an embodiment;

FIG. 3 is a flow chart illustrating in overview a method of landmark detection in accordance with an embodiment;

FIG. 4 illustrates a spline and inter-vertebrae displacement vectors in accordance with an embodiment;

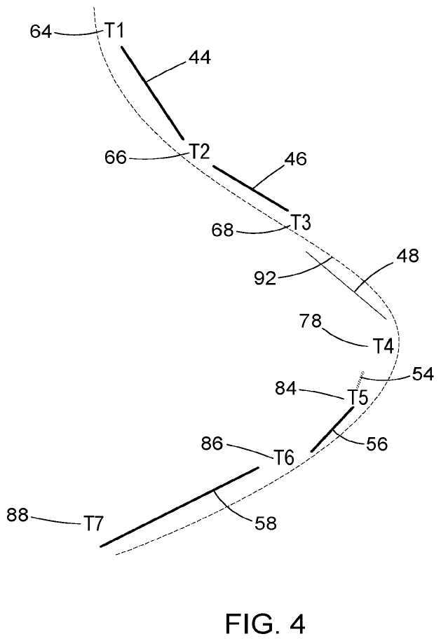

FIG. 5 illustrates spline fitting and normalisation to one dimension in accordance with an embodiment;

FIG. 6 illustrates a neural network used in some embodiments;

FIG. 7 illustrates a first part of an example convolutional neural network used in some embodiments; and

FIG. 8 illustrates a second part of an example convolutional neural network used in some embodiments.

### DETAILED DESCRIPTION

Certain embodiments provide a medical image processing apparatus comprising processing circuitry configured to obtain a respective position for each of a plurality of anatomical landmarks in a three-dimensional space, generate a spline based on the positions for the plurality of anatomical landmarks, use the spline to normalize the positions to obtain one-dimensional data or two-dimensional data, adjust at least one of the anatomical landmarks based on the one dimensional data or two-dimensional data, and re-map the adjusting of at least one anatomical landmark to the three-dimensional space.

Certain embodiments provide a medical image processing method comprising obtaining a respective position for each of a plurality of anatomical landmarks in a three-dimensional space, generating a spline based on the positions for the plurality of anatomical landmarks, using the spline to normalize the three-dimensional positions to obtain one-dimensional data or two-dimensional data, adjusting at least one of the anatomical landmarks based on the one dimensional data or two-dimensional data, and re-mapping the adjusted at least one anatomical landmark to the three-dimensional space.

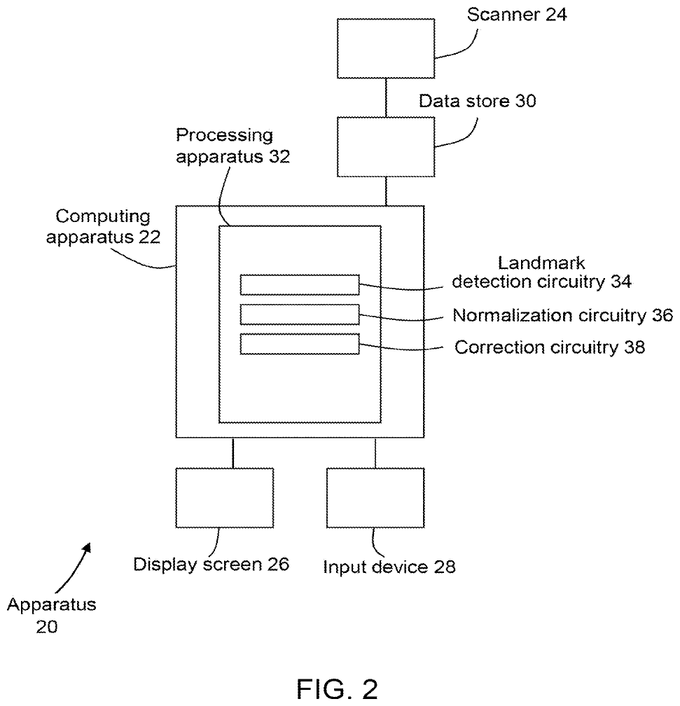

A medical image processing apparatus 20 according to an embodiment is illustrated schematically in FIG. 2. The medical image processing apparatus 20 comprises a computing apparatus 22, in this case a personal computer (PC) or workstation, which is connected to a scanner 24 and a data store 30 either directly or indirectly.

The medical image processing apparatus 20 further comprises one or more display screens 26 and an input device or devices 28, such as a computer keyboard, mouse or trackball.

In the present embodiment, the scanner 24 is a computed tomography (CT) scanner. The scanner 24 is configured to generate image data that is representative of at least one anatomical region of a patient or other subject. The image data comprises a plurality of voxels each having a corresponding data value. In the present embodiment, the data values are representative of intensity in Hounsfield units.

In other embodiments, the scanner 24 may be configured to obtain two-, three-or four-dimensional image data in any suitable imaging modality. For example, the scanner 14 may comprise a magnetic resonance (MR) scanner, computed tomography (CT) scanner, cone-beam CT scanner, positron emission tomography (PET) scanner, X-ray scanner, or ultrasound scanner.

In the present embodiment, image data sets obtained by the scanner 24 are stored in data store 30 and subsequently provided to computing apparatus 22. In an alternative embodiment, image data sets are supplied from a remote data store (not shown). The data store 30 or remote data store may comprise any suitable form of memory. In some embodiments, the medical image processing apparatus 20 is not coupled to any scanner.

Computing apparatus 22 comprises a processing apparatus 32 for processing of data. The processing apparatus comprises a central processing unit (CPU) and Graphical Processing Unit (GPU), and may further comprise a Tensor Processing Unit (TPU). Any other suitable processing circuitry may be used in other embodiments. The processing apparatus 32 provides a processing resource for automatically or semi-automatically processing medical image data sets. In other embodiments, the data to be processed may comprise any image data, which may be image data other than medical image data.

The processing apparatus 32 includes landmark detection circuitry 34 configured to detect landmarks, normalization circuitry 36 configured to normalize three-dimensional landmark positions into one dimension and adjustment circuitry 38 configured to adjust, for example correct, landmark positions in one dimension and re-map adjusted, for example corrected, positions into three dimensions.

In some embodiments the adjustment circuitry 38 may be referred to as correction circuitry.

In the present embodiment, the circuitries 34, 36, 38 are each implemented in the CPU and/or GPU and/or TPU by means of a computer program having computer-readable instructions that are executable to perform the method of the embodiment. In other embodiments, the circuitries may be implemented as one or more ASICs (application specific integrated circuits) or FPGAs (field programmable gate arrays).

The computing apparatus 22 also includes a hard drive and other components of a PC including RAM, ROM, a data bus, an operating system including various device drivers, and hardware devices including a graphics card. Such components are not shown in FIG. 2 for clarity.

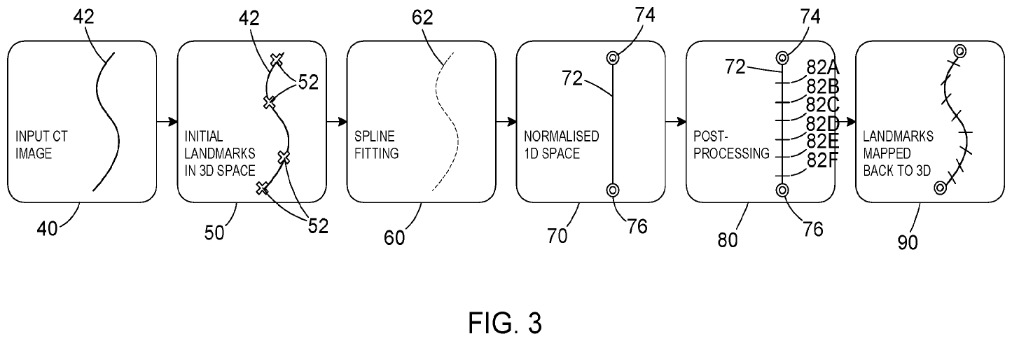

The apparatus 20 of FIG. 2 is configured to perform a method in accordance with FIG. 3. FIG. 3 is a flow chart illustrating in overview a method of landmark detection in accordance with an embodiment.

At stage 40, the landmark detection circuitry 34 receives a volumetric image data set obtained from a CT scan performed by scanner 24. The volumetric image data set may also be described as an input CT image. The volumetric image data set comprises respective intensity values for a plurality of voxels of the volumetric image data set in a coordinate space of the volumetric image data set. The coordinate space of the volumetric image data set may also be referred to as patient space. The intensity values may also be referred to as image data values. In other embodiments, the volumetric image data set may be obtained from any suitable scanner or scanners. In further embodiments, the volumetric image data set may comprise data of any suitable modality or modalities.

The CT scan from which the volumetric image data set is obtained is a CT scan of the spine of a subject. In the embodiment of FIG. 3, the CT scan of the spine includes all vertebrae of the spine. In other embodiments, the CT scan may include a sub-set of the vertebrae of the spine. For example, the CT scan may be a scan of the upper spine only, or of the lower spine only.

The spine of the subject has a natural curvature which is represented in FIG. 3 by curved line 42. Although the curvature is illustrated in two dimensions in FIG. 3, a real spine may have curvature in three dimensions. A real spine may have some side-to-side curvature as well as front-to-back curvature.

At stage 50, the landmark detection circuitry 34 performs an initial localization stage in which the volumetric image data set is processed to find the vertebrae of the spine.

In the embodiment of FIG. 3, the initial localization stage comprises inputting the volumetric image data set into a trained model that is trained to identify a plurality of anatomical landmarks. The plurality of anatomical landmarks comprises one anatomical landmark per vertebra, positioned at a centre of the vertebra. In other embodiments, different anatomical landmarks may be used.

The trained model may comprise, for example a trained convolutional neural network that is trained to output a set of output data comprising a set of points, each labelled as a respective vertebra (for example, C1 . . . C7, T1 . . . T12 and L1 . . . L3). The output data comprises a respective three-dimensional position for each point of the set of points in a coordinate space of the volumetric image data set. The three-dimensional positions may be referred to as initial landmark positions and are represented in FIG. 3 as initial landmarks 52. For example, the initial landmarks 52 may comprise the vertebrae centers in the coordinate space of the volumetric image data set. The initial landmarks 52 may also comprise a set of three dimensional positions covering part or all of the volume of vertebra. The initial landmarks 52 may also comprise a three-dimensional position for the top vertebrae 74 and the bottom vertebrae 76 which may be referred to as anchor landmarks.

As described above, it can be difficult for a trained model to correctly detect all of the vertebrae. For example, in the initial localization stage, the trained model may fail to detect one or more vertebrae along the spine, mis-label one or more vertebrae by placing labels for multiple vertebrae in a single vertebra space, or mis-label with labels being one-out from a ground truth, creating a domino effect of errors. The following stages of FIG. 3 are intended to correct incorrect data that is obtained at the initial localization stage, for example to correct incorrect labels and/or incorrect three-dimensional positions.

Any suitable adjustments, for example corrections may be performed in some embodiments. For example, the adjusting anatomical landmarks may comprise adding one or more landmarks, deleting one or more landmarks, re-ordering a sequence of the landmarks, or changing a position or label of at least one of the landmarks.

At stage 60, the normalization circuitry 36 fits a smooth three-dimensional parameterized curve to the three-dimensional points that were output at stage 52. In the embodiment of FIG. 3, the smooth three-dimensional parameterized curve is a three-dimensional spline 62. Any suitable spline fitting method may be used. For example, a low order spline may be used to fit to the centreline formed of the vertebrae centers. The spline may be fitted to the three-dimensional positions of detected initial landmarks without the need for labelled landmarks.

The fitting of the spline uses a hyperparameter that is representative of smoothness and a hyperparameter that is a measure of how well the spline goes through the three-dimensional points. Smoothness of the spline may therefore be balanced with how close the spline comes to the initial landmark positions that were output at stage 50.

At stage 60, the normalization circuitry 36 may automatically assess whether a given labelling makes sense from an anatomical perspective. Inter-vertebrae displacement vectors are a useful way to assess if a given labelling conforms to the anatomy of the spinal column. In some embodiments, the inter-vertebrae displacement vectors connect the vertebrae centers in the coordinate space of the volumetric image data set for example, the inter-vertebrae displacement vector between vertebra T8 and T9 may extend from the vertebrae center of the T8 vertebrae to the vertebrae center of the T9 vertebrae and be directed towards the upper end of the spine from T8 to T9. In other embodiments the vectors may connect other detected landmarks within the volume of each vertebrae or be directed towards an alternate detected landmark or with respect to a reference position.

The normalization circuitry 36 may compare each inter-vertebrae displacement vector (for example, each comprising a respective magnitude and direction) against the spline to assess the accuracy of a detected landmark by determining any combination of the spatial positions, the relative positions and orientations of the inter-vertebra displacement vectors. In some embodiments, this may comprise assessing the relative positions and orientations of successive landmarks, such as successive vertebra. In an embodiment, the midpoint of the displacement vector may be projected onto the spline and the tangent vector of the spline recorded at the intersection. The angle between the inter-vertebrae displacement vector and the tangent vector of the spline may be determined. In other embodiments, other metrics may be determined to assess the distance between detected landmarks and their position relative to each other in the coordinate space of the volumetric image data set.

The normalisation circuitry 36 may automatically assess the accuracy of the labelling. In one embodiment, illustrated in FIG. 3, the assessment of landmark detection accuracy is based on how accurately inter-vertebrae displacement vectors 44, 46, 48, 54, 56, 58, representing spatial arrangement of initial landmarks 52, are oriented correctly with respect to each other and are at an anatomically correct distance from each other and from detected landmarks. In some embodiments, the ground truth of orientations that the initial landmarks 52 are compared to are averages of patient records. This may be based, for example, on a reference distance between vertebra and deviation from the reference distance, or any other suitable metric. This may be based, for example, on a reference orientation in space and a deviation of the vector's orientation in space from the reference orientation. In some embodiments, determining the correct direction will be based on relative positions of successive vertebrae in a vertical axis parallel to the spinal column, whether a vertebrae is above or below its neighbouring vertebrae. In other embodiments, other measures and techniques may be used to compare a labelled volumetric image data set to an anatomically correct reference.

In FIG. 3, a two dimensional representation 92 of the three-dimensional spline 62 and a set of inter-vertebrae displacement vectors 44, 46, 48, 54, 56, 58, connecting vertebra T1 through T7 64, 66, 68, 78, 84, 86, 88 in the volumetric image data is shown. In one embodiment, the normalisation circuitry determines the inter-vertebrae displacement vectors 58, 56 connecting vertebrae T7 88 to vertebrae T6 86 and connecting vertebrae T6 86 to vertebrae T5 84 to be of correct size and with correct orientation and may record that the accuracy of landmark detection is high for T7 and/or T6 and/or T5. It may consider the inter-vertebrae displacement vector 54 connecting vertebrae T5 84 to vertebrae T4 78 to be of lower than acceptable size and record that the accuracy of landmark detection is low for vertebrae T5 and/or T4. It may for example consider the inter-vertebrae displacement vector 48 connecting vertebrae T4 78 to vertebrae T3 68 to be of a higher than acceptable size and record that the accuracy of landmark detection is low for vertebrae T4 and/or T3. It may traverse the two dimensional representation 92 of the three-dimensional spline 62 in either direction till all the inter-vertebrae displacement vectors are assessed for accuracy.

The output of stage 60 is the fitted three-dimensional spline 62.

At stage 70, the normalization circuitry 36 uses the fitted three-dimensional spline 62 to transform landmark positions from a coordinate space of the volumetric image data set to a reference coordinate space, which may also be referred to as a reference frame. The reference coordinate space is a coordinate space in which the spine appears straight and a length of the spine is normalized.

Variation in spine curvatures and orientations across imaging subjects means that useful statistical information may be lost if the spline data and the identified landmarks 52 are post-processed in patient space. In embodiments where the post-processing comprises the use of a neural network, the training set of data for the neural network may comprise this variation in spine curvatures and orientations. Transforming the fitted three-dimensional spline 62 to a lower-dimensional, for example one-dimensional, coordinate space can make it easier to train the neural network with supervised one dimensional data, or two dimensional data in some embodiments. Whilst the three dimensional spline is transformed to one-dimensional co-ordinate space in the present embodiment, in other embodiments a transformation is performed that may not result in a one-dimensional space. For example, a normal spine will exhibit most of the natural curve in the anterior-posterior direction (caused by small of back and shoulders). In some embodiments only this dimension of variation is removed, which results in a two-dimensional space.

Normalization of some sort can be beneficial in order to remove at least some, optionally as much as possible, natural, anatomical variation. This may effectively reduce the possible choices the post-processing can make to choices that preserve the anatomical variation.

Transforming the 3D space to a 1D space, or to a 2D space, is one part of the normalization, dimensionality. Length and scale are other forms of variation and those variations are also eliminated or reduced in some embodiments, By decomposing into these components, each component may be solved or determined individually and in an appropriate way, which can mean that any remaining issues are simplified. For example, in the case of a convolutional neural network (CNN), or at least some other types of network, during training the CNN can learn to detect certain features. Due to the nature of how these are defined, the features it learns may be not scale or rotation invariant (although downsampling layers may provide some degree of scale invariance). This means that if it is desired for the CNN to learn to detect features at multiple scales or angles of rotation it may have to learn to detect such features multiple times, for example at the multiple scales or angles of rotation. So, potentially the part of the CNN that can detect a vertebra of, say, 10 pixels wide may not detect it as well if it is, say, 15 pixels wide. Normalising away these variations can allow the CNN or other network to focus on other features of relevance.

In an embodiment, axial and oblique multi-planar reformatting (MPR) views are generated from the CT data or other medical imaging modalities in order to view human anatomy or other subjects. An axial or oblique MPR view may comprise an axial or oblique slice through a set of three-dimensional image data, for example a slice across the spinal column of a patient. Using curved planar reformatting (CPR) images for each vertebrae a composite image is generated from the collection of CPR image of the spine. Effectively, each vertebrae is shown in straightened form in the image. The fitted three-dimensional spline 62 is transformed to a straightened MPR view. Any other suitable views may be used in other embodiments. In some embodiments, the normalization circuitry is configured to normalize the volume data by creating a curved planar reformat using the three-dimensional spline.

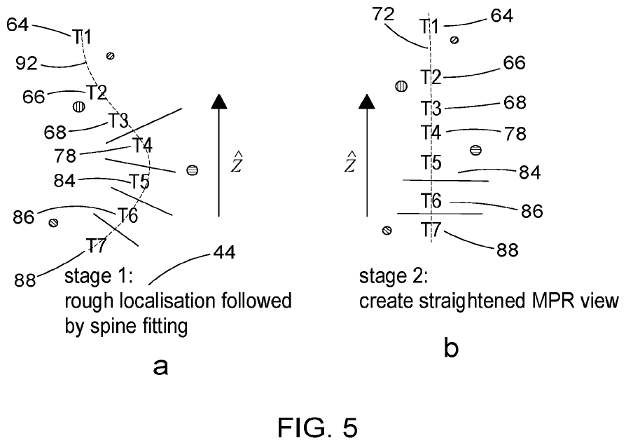

This coordinate transformation straightens the three-dimensional spline 62 to obtain a one-dimensional line 72 shown in FIG. 5 wherein the z dimension is shown vertically. Coordinates along the spline 62 are therefore shifted from three-dimensional space to one-dimensional space. A normalization is then performed in which coordinates in the one-dimensional space are scaled to a standardized length, which may also be described as a fixed size. FIG. 5n (a) shows a two dimensional representation 92 of the three-dimensional spline 62 and vertebra T1 through T7 64, 66, 68, 78, 84, 86, 88. FIG. 5(b) shows the one dimensional line 72 after the coordinate transformation with vertebra T1 through T7 labelled. In alternative embodiments the shift is from a three-dimensional space to a two-dimensional space, and the normalization is then performed in the two-dimensional space.

In the embodiment of FIG. 3, the normalization circuitry 36 determines a spine length by using the three-dimensional position, or anchor landmark, that was determined for a top vertebrae 74 at stage 50 and the three-dimensional position, or anchor landmark, that was determined for a bottom vertebrae 76 at stage 50. The top vertebra is the highest vertebra that is included in the volumetric image data set. The bottom vertebra is the lowest vertebra that is included in the volumetric image data set. For example, if the entire spine is included in the volumetric image data set, the top vertebra may be C1 and the bottom vertebra may be L5 or L6 in this example.

The normalization circuitry 36 uses the spine length determined using the positions of the top and bottom vertebrae to determine a scaling factor that is representative of a scale of the subject's spine. The scaling factor is dependent on a spine length. The normalization circuitry 36 uses the scaling factor to scale the one-dimensional space to a standardized length, thereby obtaining a normalized one-dimensional line 72.

In some circumstances, the top and bottom vertebrae may be more distinctive than other vertebrae of the spine and so may be more likely to be correctly localized in the initial localization stage.

In other embodiments, different normalizations may be used.

In some embodiments, scale is estimated by segmenting one or more of the vertebrae in the volumetric image data set. The normalization circuitry 36 determines a size or volume for each segmented vertebra and compares the determined size or volume to a standardized size or volume to determine a scaling factor. The normalization comprises using the scaling factor to transform coordinates into a one-dimensional line of a standardized length.

In some embodiments, a measurement along the spine in the volumetric image data set is used for scale. A measured length may be scaled to the standardized length.

In the embodiment of FIG. 3, the normalization circuitry 36 resamples the volumetric image data set by taking a plurality of cross-cut views perpendicular to the spline. In some embodiments, these could be CPR views of the spine. Each cross-cut view becomes a respective slice of a resampled volume. In the resampled volume, the spine is substantially straightened.

A fixed number of slices may be used, for example 50 slices or 100 slices. The resampled volume may have a fixed size, in which one dimension of the fixed size is the standardized length.

Each of the initial landmark positions is transformed into the normalized coordinate space of the resampled volume. It is expected that most of the transformed landmark positions lie on, or very near, the one-dimensional line. The reduction in spatial variation of landmark positions due to this transformation may be beneficial for post-processing at stage 80. The reduction in variations of scale and rotation in images due to the normalization may also be beneficial for the use of a neural network in the post processing stage 80.

At stage 80, the adjustment circuitry 38 applies post processing to obtain final landmark locations in the normalised coordinate space of the resampled volume. The post-processing may comprise correction of one or more landmark locations, or other adjustments. Correction may comprise addition, deletion and adjusting the one or more landmark locations or labels.

The adjustment circuitry 38 may train, and subsequently use a trained neural network to perform post-processing to obtain corrected landmark positions in normalised coordinate space. This may comprise using training data to find a mean position in normalised space. For example, in an embodiment where the normalised space comprises fewer than three dimensions, for example one vertical dimension, the training data may be used to determine a numerical mean ‘z’ value on the ‘z’-axis as illustrated in FIG. 5. Outliers can subsequently be detected by comparing landmark positions transformed in to reference space to the mean ‘z’ position using any suitable techniques, for example Gaussian likelihood, or comparison to threshold(s) e.g. based on absolute coordinates (for example, being >10 mm away from mean position is outlier in one example). Percentile thresholds could be used instead. Nearest neighbour comparison with training data may be used, as may one-class or other SVM. Any other suitable outlier detection methods may be used in other embodiments. Landmark positions transformed to reference space may also be compared to the mean ‘z’ positions of neighbouring landmarks to correct ‘one-off’ errors in labelling. Landmarks may be added or deleted or their positions adjusted This technique is referred to subsequently as ‘absolute one-dimensional mean post processing’.

Any suitable training data may be used. For example, the training data may comprise scans of spines with landmarks annotated. Using the annotated landmarks the normalisation can be applied, and mean positions may then be calculated in normalised space.

In some embodiments, absolute one-dimensional mean post processing may be used to eliminate repeated structure errors.

The adjustment circuitry 38 may perform spatial optimization of landmark positions in order to determine final landmark position. The training data or other data representative of the relevant anatomy may be used to determine coordinate points in transformed space with high predictive value of coinciding with a particular landmark. The data may be further used to determine variations in the distances between anatomical landmarks and determine a mean distance between all landmarks. The algorithm to perform spatial optimization of landmark positions may include a step wherein coordinate points in transformed space are encouraged to perform translations towards coordinate points with high predictive values. A further step in the algorithm may discourage landmark positions from being too close to each other. This technique is referred to subsequently as ‘global optimization post processing’.

In an embodiment where global optimization post processing is applied to a normalised one-dimensional space, all landmarks either translate up or down the spine or in the ±\hat{z} direction in FIG. 5(b).

Alternatively, a heatmap style approach may be used, and points may be projected to the spine spline. To expand on this point, the structure of CNNs is not immediately well suited to landmark detection. Naively, landmarks may occupy a single voxel which means the CNN may be trying to learn an extremely unbalanced problem (many negative voxels to very few positive voxels). As such, if it is desired train a CNN to identify landmarks it is known to replace the single-voxel annotations with heatmap annotations, where rather than a single positive voxel there is a Gaussian distribution, also referred to as a heatmap, of positive values with its peak at the landmark position and fading away with distance from the landmark. In some embodiments the normalisation information is used to create the CPR resampled volume and then this volume is provided to the CNN. In some other embodiments, heatmap style inference may be used, with the non-normalised volume as an input, then afterwards the predictions may be projected on to the nearest point on the spline.

The adjustment circuitry 38 may use a neural network to provide absolute one-dimensional or two-dimensional mean post processing and global optimisation post processing.

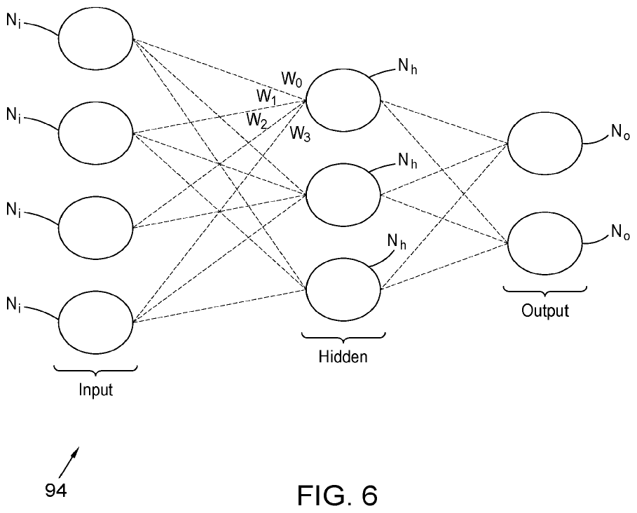

FIG. 6 shows a simplified version of a neural network 94, by way of illustrative example.

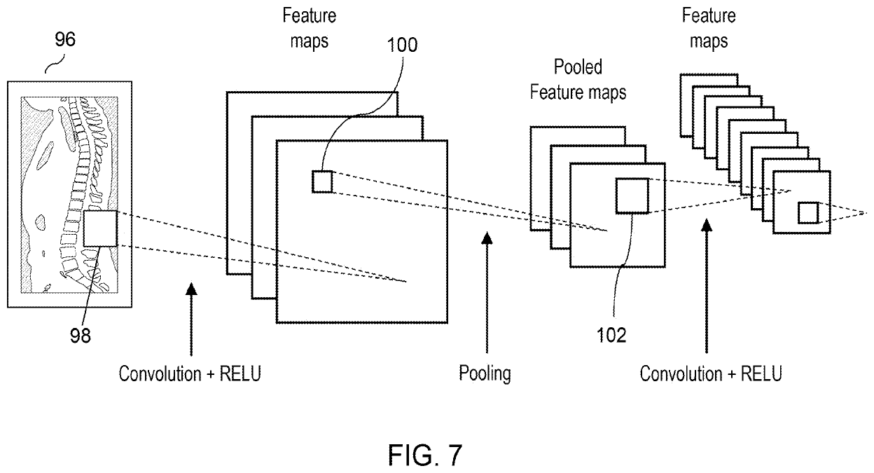

The neural network 94 comprises an input layer of nodes, a hidden layer of nodes and an output layer of nodes. In practice, there are likely to be many more nodes than those shown, and more hidden layers than the one shown. Each node of the input layer Ni receives a single value of the input data and produces at its output, an activation or node value, which is generated by carrying out an activation function (e.g. a sigmoid) on its input value. Each node Nᵢ in the input layer is connected to each node Nₕ in the hidden layer. A vector of node values from the input layer is scaled by a vector of respective weights at the input of each node in the hidden layer, each weight defining the connectivity of that particular node with its connected node in the hidden layer. The weights applied at the inputs of one of the nodes Nₕ are shown in FIG. 7 as w₀ . . . w₃. At each node Nₕ in the hidden layer, the input value at that node is given by the dot product of the weights vector connecting it to the input layer and the output values of the input nodes. The activation function is then applied to the input values at the nodes Nₕ to provide the output values of those nodes. The output vector of the hidden layer is supplied to each of the nodes in the next layer of the network (i.e. the nodes N_{O} of the output layer N_{O} in this case) and used in a similar manner to generate the output values for that next layer.

In one embodiment, for example following a Unet architecture, has 3 pooling layers before the bottleneck, with 3 unpooling layers after the bottleneck to return the spatial resolution to the same as the input resolution. At the start of the network there may be provided 32 feature maps, after each pooling layer the number of feature maps doubles for example 32, 64, 128, 256. After the bottleneck the number of features would follow the reverse pattern at each unpooling layer for example 256, 128, 64, 32. If using a 3D CNN, each kernel could have shape 3×3×3. Any other suitable model architectures may be used.

The network 94 may be trained through a variety of different methods, such as supervised or unsupervised learning. In one embodiment, the network 94 is trained through supervised leaning by determining at least one set of output values, comparing the output values to known labels representing ground truth values, and calculating an error or loss associated with the network 94 (e.g. based on a difference between the output values and the ground truth values). The loss is then back-propagated through the network 94 to update the weights, such that the network 94 is adapted to better approximate the labels from the input values. The update may optimize the weights according to an objective function (e.g. adjust the weights to reduce an error in the output values). In the next cycle, the updated weights are used with further training data to further revise the weights. In this way, the network can be trained to perform its desired operation.

When performing image classification, a convolutional neural network may be used. Convolutional neural networks (CNN) are neural networks that make use of a convolution calculation in at least one of their layers. Convolutional neural networks are particularly well adapted to image analysis and processing as they are shift invariant. However, a CNN filter is not rotation or scale invariant so it would have to learn the appearance of vertebrae multiple times to perform detection. To perform recognition of features in a series of medical images, 2D convolutional neural networks (CNN) may be applied to identify features in individual frames in series of medical images. Alternatively, 3D convolutional neural networks (CNN) may be applied to identify features, including identifying temporal relationships between frames.

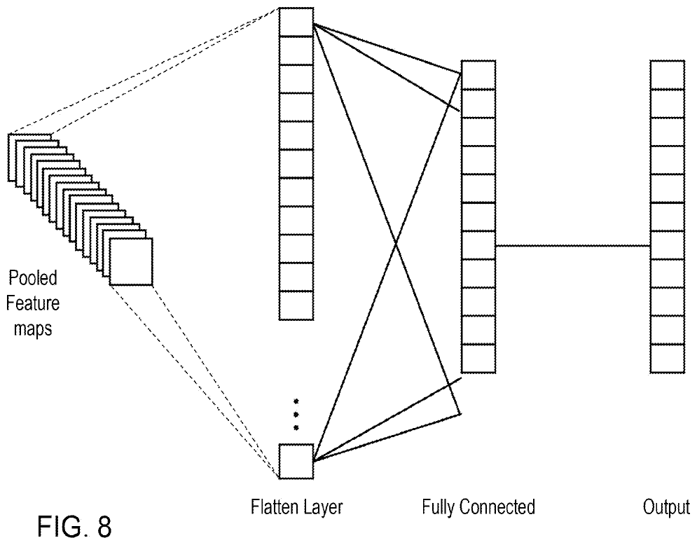

Reference is made to FIGS. 7 and 8, which illustrate an example of the operation of a convolutional neural network, which can be used to identify certain features within images and perform classification of those features. In the example shown, the input image is 96 showing the CT scan of the spine of a patient. The convolutional neural network may be used to identify where each of the vertebra is positioned.

A kernel 98 is applied to determine a convolution of the input image 96 with the kernel 98. The output of this convolution is subject to an activation function to add non-linearly. The activation function used in FIG. 7A is a rectified linear activation unit (RELU), which outputs the input if the input is positive, but outputs zero if the input is not positive. A number of feature maps 100 are generated from the input image in this way, by performing convolutions between the input image and different kernels, each of which represents a different type of basic feature, e.g. vertical line, horizontal line.

Each of the feature maps produced by the convolution and activation function is then subject to a pooling process, which is performed to reduce the spatial size of the convolved feature. The pooling process involves translating a kernel across the feature map to sample groups of pixels and returning the maximum or average value from each of the sampled pixels in the feature map. The resulting pooled feature maps are each subject to a further convolutions process (with the RELU function applied) using a respective kernel to generate a further set of feature maps from which pooling is again performed.

The pooled feature maps resulting from multiple stages of convolution and pooling may be used to produce a one dimensional array, which is provided as an input to a feed forward neural network. The resulting output values represent the locations of vertebra in the CT-scan image. The system 104 may process the output values to infer whether or not the locations of vertebra are correct.

The convolutional neural network may be trained by comparing output values for different images to labels of those images and adjusting the weights of the feed forward portion of the convolutional neural network.

The normalisation carried out in step 70 can be beneficial for the operation of the CNN because it removes variations in scale and rotation. Other types of analysis can use landmark positions that have reduced inter-patient variance. In such analyses, post-processing can be implemented using probabilistic modelling of landmark positions, such as principal component analysis or Gaussian mixture models for different vertebrae. The detection of misplaced landmarks may also be used to identify certain pathologies where this can be expected, such as age related and degenerative condition. Any suitable pathologies may be identified, for example degenerative disk disease or scoliosis.

At stage 90, the adjustment circuitry 38 maps the final landmark locations from the normalised coordinate space into the coordinate space of the volumetric image data set. The output is a series of coordinate points that represent anatomical landmarks in the volume of the patient scanned.

Techniques described herein may be applied to any suitable subject, for example any suitable human or animal subject.

In various embodiments any other suitable landmarks may be used, for example ribs or teeth.

Although the use of a neural network, for example a CNN, has been described, any other suitable trained model may be used in other embodiments, for example for registration, post-processing, adjustment, normalisation or any other suitable stage of the process. For instance, Graph Convolutional Neural Networks or transformer architectures may be used in some embodiments.

Any suitable adjustments, for example corrections, of landmarks may be performed in some embodiments. For example, the adjusting of anatomical landmarks may comprise adding one or more landmarks, deleting one or more landmarks, re-ordering a sequence of the landmarks, or changing a position or label of at least one of the landmarks, for example by application of a suitable trained model or other suitable processing steps.

In some embodiments, the or a normalised volume may be used for further processing, and for example scaling may be performed in three dimensions. For example, segmentation of a single vertebrae may be performed and then normalised across all three dimensions. It is a feature that further processing can be more effective in the normalised space.

Absolute 1D mean post-processing to eliminate repeated structure errors can be used in some embodiments. Training data can be used to find a mean z-position in normalised space. Outliers at inference time can be replaced with the mean z value for that landmark. Outliers are detected by comparing predicted landmark positions to the mean z-position (e.g. using Gaussian likelihood). Comparing predicted landmark position to the neighbour landmark mean can be used for correction, for example to detect and correct a one-off domino effect.

A global optimisation post-processing can be used to calibrate final positions in some embodiments. For example, one term can be used to encourage points to locations with high predictive values. Another, repulsive, term can be used to discourage landmarks for being too close in relative position. In 1D space, this may ensure all landmarks are pushed up/down spine spline.

One or more CNNs may be used in some embodiments. A CNN filter is not rotation or scale invariant so it would usually have to learn the appearance of vertebrae multiple times. The normalisation attempts to remove rotation or scale variance meaning CNNs can be even more effective.

Certain embodiments provide a medical image processing apparatus comprising processing circuitry configured to: receive volume data including a plurality of vertebrae, detect a plurality of anatomical landmarks corresponding to the plurality of vertebrae, generate a three-dimensional spline based on the plurality of anatomical landmarks, normalize the three-dimensional spline to one-dimensional data, correct at least one of the anatomical landmarks based on the one-dimensional data, re-map the corrected anatomical landmarks to three-dimensional space.

The processing circuitry may be further configured to: correct the at least one of the anatomical landmarks based on a supervised data, wherein the supervised data is data wherein the plurality of anatomical landmarks and positional information is correlated.

The processing circuitry may be further configured to: correct the at least one of the anatomical landmarks on the one-dimensional data, wherein the one-dimensional data is normalized based on anchor landmarks.

Certain embodiments provide a medical image processing apparatus comprising processing circuitry configured to:
Receive an image of the spine
Perform initial detection of vertebrae landmarks
Detect parameters of spine variation in the image by:
   Estimating the curve of the spine by fitting a spline curve to the detected vertebrae
   Estimating scale of the spine
Use the detected parameters to normalise the image and landmarks data
Apply normalisation-boosted post-processing to the vertebrae landmarks using:
   Absolute 1D mean to find a mean z-position in the normalised space. This normalised representation of the image and/or landmarks leads to improved accuracy of the landmark locations
   Global optimisation post-processing to calibrate final positions using terms to encourage location prediction and discourage proximity of neighbouring landmarks
   CNN heatmap detection approach where landmark positions are projected to spine spline.

The scale parameters may be estimated by measuring the distance between top and bottom vertebrae.

The scale parameters may be estimated by segmenting a vertebrae and measuring size or volumetric properties of the segmentation.

The normalisation of the image data may be done by creating a curved planar reformat using the fitted spline curve.

The normalisation of the landmarks may be done by transforming the 3D coordinates to a 1D coordinate which determines the position along the length of the spline curve.

Whilst particular circuitries have been described herein, in alternative embodiments functionality of one or more of these circuitries can be provided by a single processing resource or other component, or functionality provided by a single circuitry can be provided by two or more processing resources or other components in combination. Reference to a single circuitry encompasses multiple components providing the functionality of that circuitry, whether or not such components are remote from one another, and reference to multiple circuitries encompasses a single component providing the functionality of those circuitries.

Whilst certain embodiments have been described, these embodiments have been presented by way of example only, and are not intended to limit the scope of the invention. Indeed the novel methods and systems described herein may be embodied in a variety of other forms. Furthermore, various omissions, substitutions and changes in the form of the methods and systems described herein may be made without departing from the spirit of the invention. The accompanying claims and their equivalents are intended to cover such forms and modifications as would fall within the scope of the invention.

1. A medical image processing apparatus comprising processing circuitry configured to:
obtain a respective position for each of a plurality of anatomical landmarks in a three-dimensional space;
generate a spline based on the positions for the plurality of anatomical landmarks;
use the spline to normalize the positions to obtain one-dimensional data or two-dimensional data;
adjust at least one of the anatomical landmarks based on the one dimensional data or two-dimensional data; and
re-map the adjusting of at least one anatomical landmark to the three-dimensional space.

2. A medical image processing apparatus according to claim 1, wherein the plurality of anatomical landmarks correspond to a plurality of vertebrae.

3. A medical image processing apparatus according to claim 2, wherein the processing circuitry is further configured to receive volume data including the plurality of vertebrae, and to detect the plurality of anatomical landmarks in the volume data, thereby to obtain the positions of the anatomical landmarks.

4. A medical image processing apparatus according to claim 3, wherein the processing circuitry is further configured to normalize the volume data by creating a curved planar reformat using the three-dimensional spline.

5. A medical image processing apparatus according to claim 3, wherein the detecting of the plurality of anatomical landmarks comprises automated landmark detection using a trained model.

6. A medical image processing apparatus according to claim 1, wherein the adjusting of the one or more anatomical landmarks is based on supervised data, wherein the supervised data is data that the plurality of anatomical landmarks and positional information is correlated.

7. A medical image processing apparatus according to claim 1, wherein the adjusting of the one or more anatomical landmarks comprises comparing the one-dimensional data or two-dimensional data to a set of one-dimensional or two-dimensional reference positions and adjusting the one-dimensional data or two-dimensional data based on the one-dimensional or two-dimensional reference positions.

8. A medical image processing apparatus according to claim 7, wherein the one-dimensional or two-dimensional reference positions are mean positions for the plurality of anatomical landmarks in a reference data cohort.

9. A medical image processing apparatus according to claim 1, wherein the adjusting of the one or more anatomical landmarks comprises using a calibration to encourage location prediction and discourage proximity of neighbouring landmarks.

10. A medical image processing apparatus according to claim 4, wherein processing circuitry is configured to use at least one neural network or other trained model to at least one of: perform a registration process to obtain the landmark positions; perform the normalisation; or perform the adjusting or remapping of the at least one anatomical landmark.

11. A medical image processing apparatus according to claim 1, wherein the normalizing of the three-dimensional positions comprises estimating a scale and normalizing in dependence on the estimated scale.

12. A medical image processing apparatus according to claim 11, wherein the estimating of the scale is based on positions of two or more anchor landmarks.

13. A medical image processing apparatus according to claim 12, wherein a first anchor landmark corresponds to a top spinal vertebra and a second anchor landmark corresponds to a bottom spinal vertebra.

14. A medical image processing apparatus according to claim 3, wherein the processing circuitry is further configured to segment one or more anatomical structures in the volume data and to determine a size or volume of the segmented one or more anatomical structures, and wherein the normalizing of the three-dimensional positions comprises estimating a scale based on the determined size or volume and normalizing in dependence on the estimated scale.

15. A medical image processing apparatus according to claim 1, wherein the spline comprises a three-dimensional spline.

16. A medical image processing apparatus according to claim 1, wherein the adjusting of the at least one of the anatomical landmarks comprises correcting the at least one of the anatomical landmarks.

17. A medical image processing apparatus according to claim 1, wherein the adjusting of the at least one of the anatomical landmarks comprises adding at least one of the landmarks, deleting at least one of the landmarks, re-ordering a sequence of the landmarks, or changing a position or label of at least one of the landmarks.

18. A medical image processing apparatus according to claim 1, wherein the processing circuitry is further configured to determine at least one pathology based on the one-dimensional data or two-dimensional data.

19. A medical image processing apparatus according to claim 1, wherein the plurality of anatomical landmarks correspond to a plurality of vertebrae, ribs or teeth.

20. A medical image processing method comprising:
obtaining a respective position for each of a plurality of anatomical landmarks in a three-dimensional space;
generating a spline based on the positions for the plurality of anatomical landmarks;
using the spline to normalize the three-dimensional positions to obtain one-dimensional data or two-dimensional data;
adjusting at least one of the anatomical landmarks based on the one dimensional data or two-dimensional data; and
re-mapping the adjusted at least one anatomical landmark to the three-dimensional space.
